(19) United States
(12) Patent Application Publication
(10) Pub. No.: US 2024/0002788 A1
(43) Pub. Date: Jan. 4, 2024

(54) Regenerative Tissue Extract from Non-Human Mammalian Placenta

(71) Applicant: VityleBio, Inc., Gainesville, FL (US)

(72) Inventors: Shannon Brown Fedenko, Gainesville, FL (US); Isaac Finger-Baker, Gainesville, FL (US)

(21) Appl. No.: 18/039,372

(22) PCT Filed: Dec. 3, 2021
(86) PCT No.: PCT/US21/72737

§ 371 Date: May 30, 2023

Related U.S. Application Data
(60) Provisional application No. 63/120,823, filed on Dec. 3, 2020.

(51) Int. Cl. C12N 5/073; A61P 17/02; A61P 19/00

(52) U.S. Cl. CPC C12N 5/0605; A61P 17/02; A61P 19/00; C12N 2509/10

(57) ABSTRACT

Disclosed herein are embodiments of an invention relating to non-human mammalian placental extract compositions, methods of extraction of compositions from non-human mammalian placenta, and methods of treatment using non-human mammalian placental compositions. These compositions have regenerative properties for medical and veterinary applications like tissue and cell culture and regeneration. These regenerative properties include anti-inflammatory, anti-oxidative, anti-microbial, pro-osteogenic, pro-angioenic, pro-neurogemc, pro-neuronal, and immune modulating, The present invention allows for effective bioburden mitigation and extraction of from non-human mammalian placenta and placenta collected in non-clinical environments for medical and veterinary applications.

This application claims priority to U.S. Provisional Application No. 63/120,823, which is incorporated in its entirety by reference.

### BACKGROUND

The present invention relates to purified non-human mammalian placental extract compositions with regenerative properties. The compositions have been found to have regenerative properties for medical and veterinary applications. Regenerative properties include anti-inflammatory, anti-oxidative, anti-microbial, pro-osteogenic, pro-angiogenic, pro-neurogenic, pro-neuronal, and immune modulating.

Mammalian placenta can be classified in certain groups including cotyledonary (e.g., cow, sheep, goat), discoid (e.g., human, mouse), diffuse (e.g., pig, horse, camel), and zonary (dog, cat).

Current technologies do not address the extraction of compositions from non-human placenta for use in medical applications. Non-human placenta is different from human placenta in protein makeup, lipid levels, hormones, levels of blood and other materials, and structure and subsections, such as whether amnion is included. Purified non-human placental extract compositions have additional therapeutic potential for use in medical and veterinary applications.

Current technologies collect human placenta, often from cesarean section procedures. Human placenta is relatively clean as a starting material and possesses a relatively low bioburden. Non-human placenta collected in non-clinical environments inherently possesses high bioburden levels. Bioburden mitigation is essential for medical and veterinary applications—to reduce the risk of passing on a deleterious foreign organism to the recipient of the extract. The present invention allows for effective bioburden mitigation and extract of tissue from non-human placenta for medical and veterinary applications.

Other features of the present invention will be apparent to those of ordinary skill in the art in light of this disclosure.

### SUMMARY

Embodiments of the disclosed invention may comprise purified placental extract compositions, methods of making a purified placenta extract compositions, methods of treatment using placental extract compositions, methods of inducing regenerative effects and cell proliferation using placental extract compositions. Inventive compositions include a composition that is the result of the following steps: obtaining cotyledonary placenta; decontaminating the placenta; mechanically disrupting the placenta; placing the mechanically disrupted placenta in an ionic buffer with a protease neutralizing reagent to create a protein precipitate; isolating and retaining the solid phase of the protein precipitate; incubating the solid phase in a linearization buffer to linearize proteins; isolating the soluble proteins by phase separation; and purifying the liquid phase with soluble proteins by removing the linearization buffer to obtain a purified composition. In certain embodiments, the cotyledonary placenta is bovine placenta, ovine placenta, or caprine placenta. In certain embodiments the purified composition comprises between 647 and 740 unique proteins. In certain embodiments, the two most prevalent protein clusters in the purified composition are vimentin and actin. In certain embodiments, decontaminating the placenta comprises chemical treatment with water, bleach, iodine, isopropanol, sterile buffers, or a combination thereof, from which the washate is separated from the placenta via macropore filtration. In certain embodiments, mechanically disrupting the placenta comprises grinding, chopping, cryo-milling, blending into pieces less than or equal to four square centimeters (=4 cm²), probe homogenization, or a combination thereof. In certain embodiments, the ionic buffer comprises sodium chloride, tris, phosphate buffered saline, saline, salt buffers with a molarity greater than or equal to three molar (≥3M), or a combination thereof. In certain embodiments, placing the solid phase in a linearization buffer comprises incubating the solid phase with mechanical agitation at four degrees Celsius (4° C.). In certain embodiments, the linearization buffer comprises urea, sodium dodecyl sulfate, chaotropic agents, detergents in buffers, or a combination thereof. In certain embodiments, phase separation comprises centrifugation, filtration, or a combination thereof. In certain embodiments, purifying the liquid phase with soluble proteins comprises dialyzing against a sterile buffer lacking linearizing reagents at four degrees Celsius (4° C.) for at least twelve cumulative hours (12 hr), dialyzing with a molecular weight cutoff of at least one kilo-Dalton (1 kDa), tangential flow filtration, or a combination thereof. In certain embodiments, the steps for making the composition further comprise sterilizing the composition to achieve a bioburden reduction of at least one log (1 log) via addition of a chemical sterilant, filtration, energy or particle irradiation, or a combination thereof. In certain embodiments, the steps for making the composition include drying the composition by lyophilizing, powderizing the composition by cryo-milling, or a combination thereof. In certain embodiments, the steps for making the composition are conducted in a controlled environment of at least ISO level 8.

Inventive methods of making a composition include a method comprising: obtaining cotyledonary placenta; decontaminating the placenta: mechanically disrupting the placenta; placing the mechanically disrupted placenta in a highly ionic buffer with a protease neutralizing reagent to create a protein precipitate; isolating and retaining the solid phase of the protein precipitate; placing the solid phase in a linearization buffer to linearize proteins; isolating the soluble proteins by phase separation; and purifying the liquid phase with soluble proteins by removing the linearization buffer to obtain a purified composition. In certain embodiments, the cotyledonary placenta is bovine placenta, ovine placenta, or caprine placenta. In certain embodiments the purified composition comprises between 647 and 740 unique proteins. In certain embodiments, the two most prevalent protein clusters in the purified composition are vimentin and actin. In certain embodiments, decontaminating the placenta comprises chemical treatment with water, bleach, iodine, isopropanol, sterile buffers, or a combination thereof, from which the washate is separated from the placenta via macropore filtration. In certain embodiments, mechanically disrupting the placenta comprises grinding, chopping, cryo-milling, blending into pieces less than or equal to four square centimeters (≤4 cm²), probe homogenization, or a combination thereof. In certain embodiments, the ionic buffer comprises sodium chloride, tris, phosphate buffered saline, saline, salt buffers with a molarity greater than or equal to three molar (≥3M), or a combination thereof. In certain embodiments, placing the solid phase in a linearization buffer comprises incubating the solid phase with mechanical agitation at four degrees Celsius (4° C.). In certain embodiments, the linearization buffer comprises urea, sodium dodecyl sulfate, chaotropic agents, detergents in buffers, or a combination thereof. In certain embodiments, phase separation comprises centrifugation, filtration, or a combination thereof. In certain embodiments, purifying the liquid phase with soluble proteins comprises, dialyzing against a sterile buffer lacking linearizing reagents at four degrees Celsius (4° C.) for at least twelve cumulative hours (12 hr), dialyzing with a molecular weight cutoff of at least one kilo-Dalton (1 kDa), tangential flow filtration, or a combination thereof. In certain embodiments, the steps for making the composition further comprise sterilizing the composition to achieve a bioburden reduction of at least one log (1 log) via addition of a chemical sterilant, filtration, energy or particle irradiation, or a combination thereof. In certain embodiments, the steps for making the composition include drying the composition by lyophilizing, powderizing the composition by cryo-milling, or a combination thereof. In certain embodiments the steps for making the composition are conducted in a controlled environment of at least ISO level 8.

Inventive methods of treatment include a method of treating a broken bone, burn, wound, or lesion in a mammal comprising administering to the subject mammal a composition comprising a purified cotyledonary placental extract. In certain embodiments, purified coytledonaly placental extract is a purified bovine placental extract, a purified ovine placental extract, or a purified caprine placental extract. In certain embodiments, the purified bovine placental extract comprises between 647 and 740 unique proteins. In certain embodiments, the two most prevalent protein clusters in the purified bovine placental extract are vimentin and actin. In certain embodiments, the subject mammal is a human or a canine.

Inventive methods can also include a method of inducing regenerative effects in a mammal comprising administering to the subject mammal a composition comprising a purified cotyledonary placental extract. In certain embodiments, purified coytledonaly placental extract is a purified bovine placental extract, a purified ovine placental extract, or a purified caprine placental extract. In certain embodiments, the purified bovine placental extract comprises between 647 and 740 unique proteins. In certain embodiments, the two most prevalent protein clusters in the purified bovine placental extract are vimentin and actin. In certain embodiments, the subject mammal is a human or a canine.

Inventive methods can also include a method of inducing cell proliferative effects in a mammal comprising administering to the subject mammal a composition comprising a purified cotyledonary placental extract. In certain embodiments, purified coytledonaly placental extract is a purified bovine placental extract, a purified ovine placental extract, or a purified caprine placental extract. In certain embodiments, the purified bovine placental extract comprises between 647 and 740 unique proteins. In certain embodiments, the two most prevalent protein clusters in the purified bovine placental extract are vimentin and actin. In certain embodiments, the subject mammal is a human or a canine.

### BRIEF DESCRIPTION OF THE DRAWINGS

For purpose of explanation, several embodiments are set forth in the following figures, wherein:

FIG. 1 is a block diagram depicting methods of making a placental extract composition.

FIG. 2 are pictures from an in vitro investigation of a placental extract composition.

FIG. 3 are pictures of the morphology of cells for placental extract composition coated wells.

FIG. 4 are graphical representations of results from anti-oxidative capacity testing of a placental extract composition.

FIG. 5 is a graphical representation of results from testing involving the conversion of PrestoBlue.

FIG. 6 is a graphical representation of results from testing of antioxidative stability.

FIG. 7 is a graphical representation of results of bioburden reduction.

FIG. 8 are graphical representations of physical properties of embodiments of a placental extract composition.

FIG. 9 are pictures demonstrating the healing effects of an embodiment of a placental extract composition.

FIG. 10 are pictures demonstrating the healing effects of an embodiment of a placental extract composition.

FIG. 11 are pictures demonstrating the healing effects of an embodiment of a placental extract composition.

FIG. 12 are results from mass spectroscopy analysis of placental extract compositions.

### DESCRIPTION OF THE EMBODIMENTS

In the following description, details are set forth for purpose of explanation. The embodiments (and descriptions) disclosed herein are intended, therefore, to be illustrative only and not limiting. Similarly, where examples are used herein, the examples are not intended to be limiting unless the context in which the example is used clearly indicates otherwise. Accordingly, “for example” or “e.g.” should be read as “for example, and without limitation,” unless the context indicates that limitation to the given example(s) is intended.

The meaning of certain terms may be defined herein or will otherwise be apparent to those of ordinary skill as the ordinary meanings used in the art.

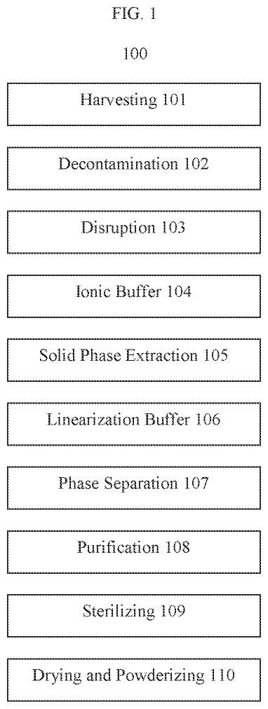

FIG. 1 depicts steps to a method of extracting a purified composition from a non-human mammalian placenta and preparation of a composition for use in the regeneration of tissue. Process 100 comprises harvesting a non-human mammalian placenta (NHMP) 101, decontamination of the NHMP 102, disruption of the NHMP 103, addition of an ionic buffer to the NHMP material 104, solid phase extraction of the buffer/NHMP material mixture 105, addition of linearization buffer to the solid phase extraction 106, phase separation of the linearization buffer/solid phase mixture 107, and purification of the liquid phase of linearization buffer/solid phase mixture 108. In certain embodiments, process 100 further comprises sterilizing 109, drying and powderizing 110, or a combination thereof.

In certain embodiments the NHMP is harvested from bovine, equine, porcine, ovine, or caprine sources. In certain preferred embodiments the NHMP that is harvested is cotyledonary placenta. In certain preferred embodiments, decontamination 102 of the NHMP comprises chemical treatment with water, bleach, iodine, isopropanol, sterile buffers, or a combination thereof, from which the washate is separated from the placenta via macropore filtration. The decontamination 102 allows for bioburden mitigation necessary for medical and veterinary applications. In certain preferred embodiments, disruption 103 of the placenta comprises grinding, chopping, cryo-milling, blending into pieces less than or equal to four square centimeters (≥4 cm²), probe homogenization, or a combination thereof. The disruption 103 allows for extraction with minimal debris and bioburden mitigation necessary for medical and veterinary applications. In certain preferred embodiments, use of an ionic buffer 104 comprises placing the disrupted NHMP in an ionic buffer with a protease neutralizing reagent to create a protein precipitate, wherein such buffer comprises sodium chloride, tris, phosphate buffered saline, saline, salt buffers with a molarity greater than or equal to three molar (≥3M), or a combination thereof. The use of a protease neutralizing reagent allows for preservation of proteins in the disrupted NHMP that contribute to the regenerative properties of the extracted composition. In certain preferred embodiments, step 105 can be isolating and retaining the solid phase of the protein precipitate from step 104. In certain preferred embodiments, use of linearization buffer 106 comprises incubating the solid phase from 105 with a linearization buffer to linearize proteins and mechanically agitating the mixture at four degrees Celsius (4° C.). In certain preferred embodiments, a linearization buffer that is useful for step 106 comprises urea, sodium dodecyl sulfate, chaotropic agents, detergents in buffers, or a combination thereof. In certain preferred embodiments, the next step 107 is isolating the soluble proteins by phase separation of the mixture from 106 comprising centrifugation, filtration, or a combination thereof and separation of the solid and liquid phase. The next step is purification 108 of the liquid phase of the mixture from 107. In certain preferred embodiments, purification 108 comprises removing the linearization buffer, dialyzing against a sterile buffer lacking linearizing reagents at four degrees Celsius (4° C.) for at least twelve cumulative hours (12 hr), dialyzing with a molecular weight cutoff of at least one kilo-Dalton (1 kDa), tangential flow filtration, or a combination thereof. Purification 108 can be continuous (for example, twelve continuous hours) or interrupted and can be for varying lengths of time. Steps 104, 105, 106, 107, and 108 allow for isolation of a composition with certain proteins to induce regenerative effects in mammals. In certain preferred embodiments, sterilizing 109 of the purified mixture from 108 comprises achieving a bioburden reduction of at least one log (1 log) via addition of a chemical sterilant, filtration, energy or particle irradiation, or a combination thereof. Sterilization 109 allows for additional bioburden mitigation that may be necessary for certain medical and veterinary applications. In certain preferred embodiments, drying and powderizing 110 the composition from 109 comprises drying the composition by lyophilizing, powderizing the extract by cryo-milling, or a combination thereof. Drying and powderizing 110 allows for an composition that can be delivered in convenient and useful media formats, such as liquid, solid, or powder, to be injectable, applied easily to external wounds, or combined with other therapeutics. In certain preferred embodiments, the steps of process 100 are conducted in a controlled environment of at least International Organization for Standardization (ISO) level 8.

Compositions obtained from embodiments of the invention such as process 100, have been found to have regenerative properties for medical and veterinary applications.

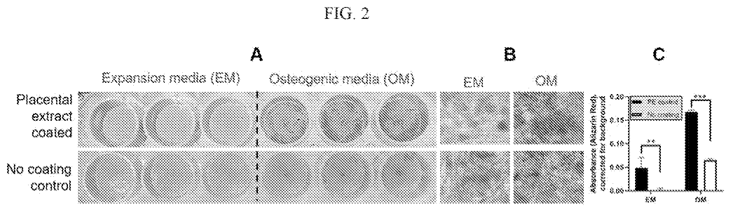

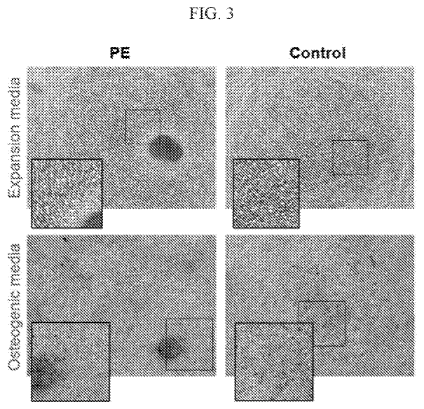

Referring to FIGS. 2 and 3, compositions obtained from embodiment of the invention, such as process 100, have been found to have pro-osteogenic effect desirable in regenerative medicine and related methods of treatment.

FIG. 2 depicts in an in vitro investigation of an placental extract composition (PE)-induced mineralization where tissue culture treated well plates were pre-coated with a thin layer of PE or left uncoated. FIG. 2 shows (A) a macroscopic photograph of the wells post staining with Alizarin Red, (B) micrograph of representative wells at 5× magnification, and (C) quantification of Alizarin Red (mineralization). (** p<0.01, *** p<0.001 via 2-way ANOVA with a Tukey's HSD. (n=3) The example placental extract compositions used in the in experiments depicted in FIG. 2 were obtained from process 100 wherein a bovine (cotyledonary) placenta was harvested in step 101. MG-63 cells were seeded onto the wells and were cultured in either standard expansion media (negative control) or in osteogenic media, with media changes every 3-4 days. After 3 weeks, the wells were stained with Alizarin Red S, which stains red for calcium deposits, as an indication of a pro-osteogenic phenotype. Mineralization was quantified by absorbance measurements of Alizarin Red S. Compared to uncoated wells, wells initially coated with PE had more staining and calcium content and, therefore, a greater osteogenic effect.

Osteogenesis/osteoinduction, the transition of non-bone cells to bone-like cells, is just one aspect to bone healing. Placental extract compositions prepared according to embodiments invention shows beneficial effects in helping bones and other healing mechanisms, such as stimulating native osteoblasts to increase mineralization, recruiting cells from the blood/marrow/periosteum, and facilitating vascularization. For example, FIG. 3, shows the morphology of cells at 3 weeks of culture, at 5× magnification. The images for the placental extract composition-coated wells in FIG. 3 have clear cellular organization. The example placental extract compositions used in the investigation depicted in FIG. 3 were obtained from process 100 wherein a bovine (cotyledonary) placenta was harvested in step 101.

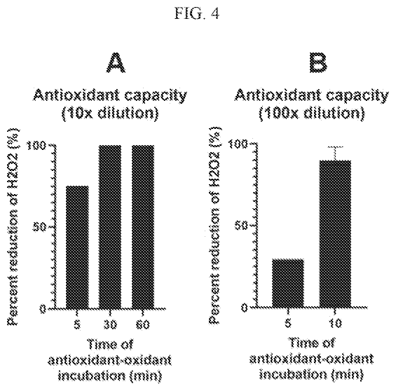

Referring to FIGS. 4-6, compositions obtained from embodiments of the invention such as process 100 have also been found to be anti-oxidative.

Referring to FIG. 4, oxidative stress in tissues and cells can promote pathogenesis, and mitigation of oxidative stress via the administration of anti-oxidants is a potential pathway to disease management. The anti-oxidative capacity of placental extract compositions (PE) according to the invention were demonstrated by observing hydrogen peroxide (H₂O₂) reduction via the Amplex Red Assay. Two dilutions of PE were mixed 1:1 with 100 μM of H₂O₂ (a reactive oxygen species) for 5, 10, or 30 min. The reduction of H₂O₂ was measured relative to a pure H₂O₂ control lacking PE. All concentrations and incubation times illustrate an anti-oxidative capacity of embodiments of the inventive PE compositions. Antioxidative effects (partial or complete reduction of H₂O₂) were observed at both (A) a 10× dilution and (B) a 100× dilution of PE. (n=8 for 10 min, n=1 for all other incubation times). The example placental extract compositions used in the experiments depicted in FIG. 4 were obtained from process 100 wherein a bovine (cotyledonary) placenta was harvested in step 101.

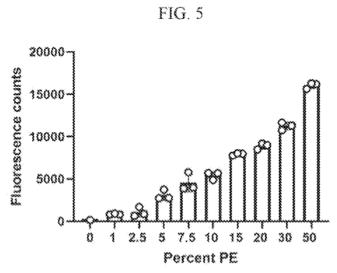

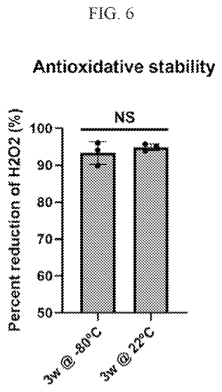

Referring to FIGS. 5 and 6, the antioxidant nature of the inventive placental extract compositions (PE) suggests it could be beneficial to combat disease influenced by oxidative stress. Such applications include liver disease, diabetic complications, myocarditis, renal disease, etc. The antioxidant properties of PE are evident from having studied the physical properties of PE. For example, FIG. 5 shows PE can also convert PrestoBlue, a resazurin reagent thought to be converted to resorufin (a fluorescent molecule) through reduction. PE has the ability to facilitate this redox reaction, where more PE results in more reagent conversion. The ability to contribute to a redox reaction via reduction, supports the idea that PE can combat oxidative environments through redox. FIG. 6 also shows PE maintains its antioxidative nature even after storage at room temperature for 3 weeks. The tests conditions for the antioxidant stability experiments were 10 minutes of placental extract incubation with 100 μM H₂O₂. Quantified remaining 11202 via Amplex Red. NS=not significant by a 2-tailed unpaired t-test (p>0.05). The example placental extract compositions used in the in the experiments depicted in FIGS. 5 and 6 were obtained from process 100 wherein a bovine (cotyledonary) placenta was harvested in step 101.

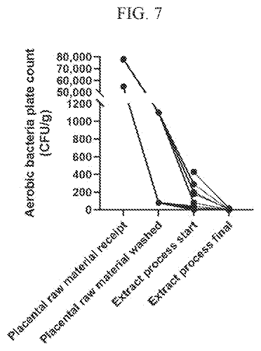

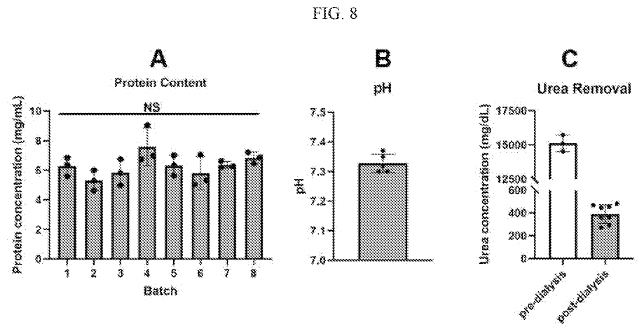

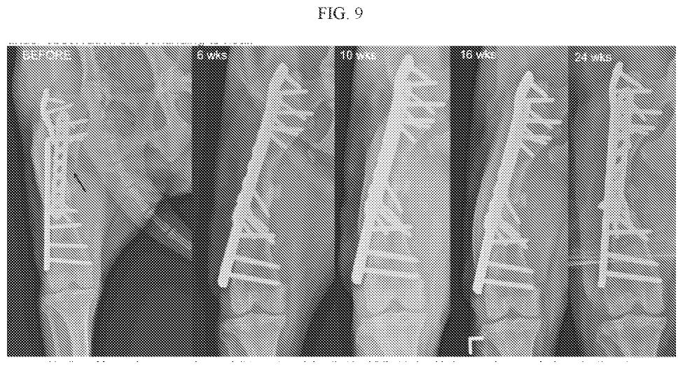

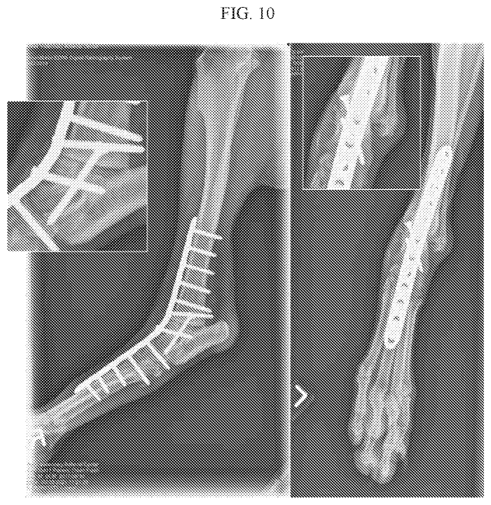

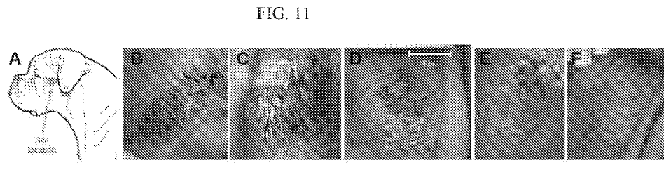

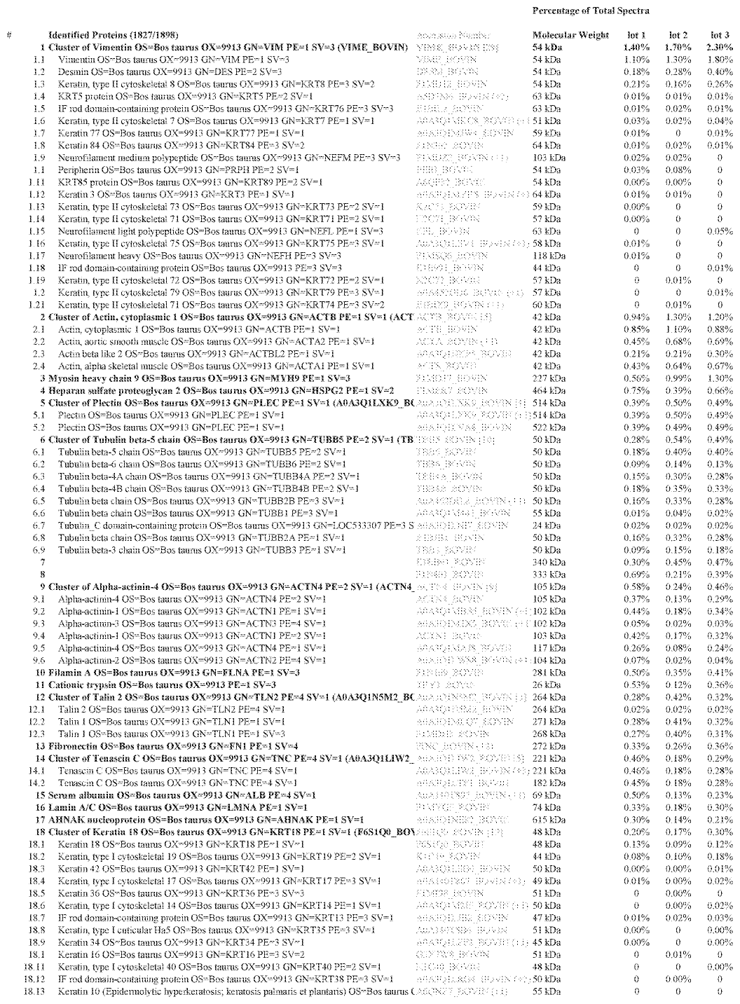

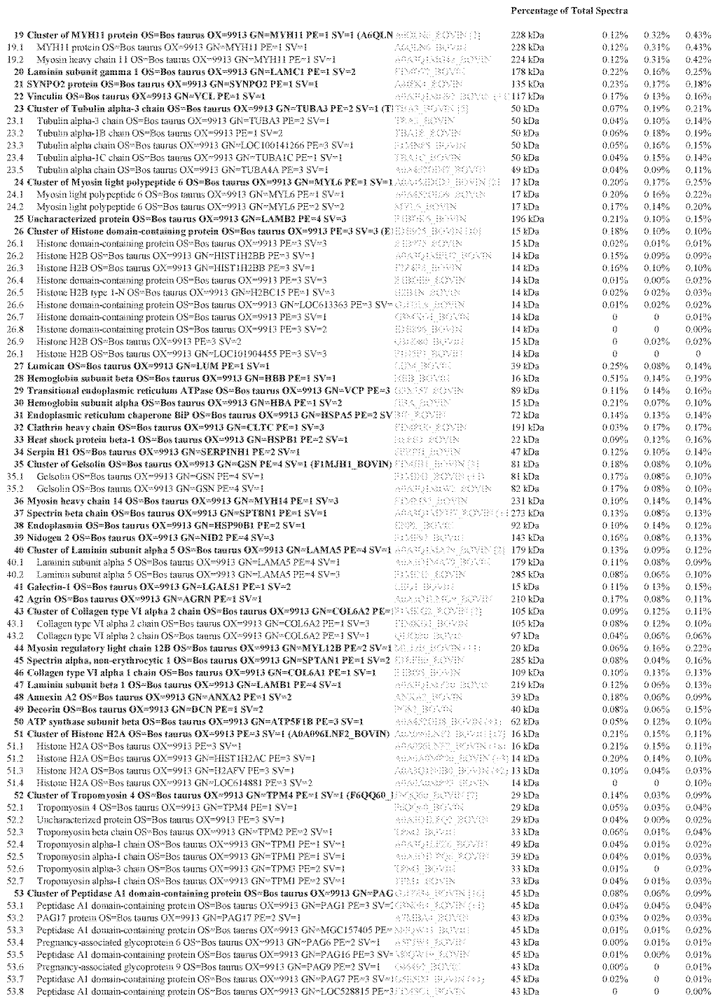

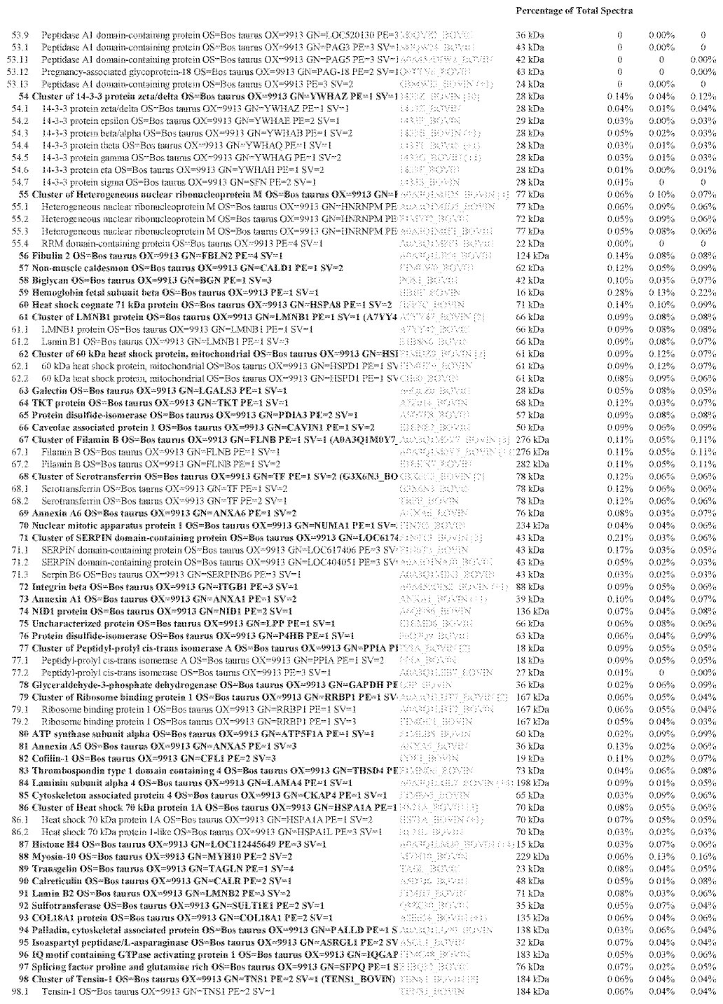

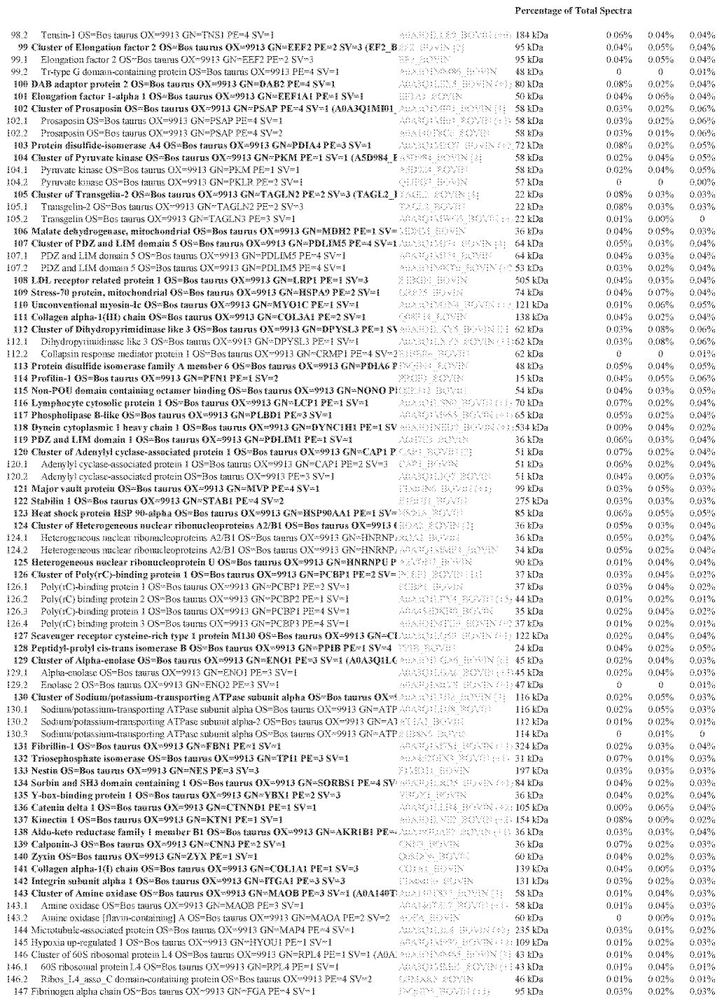

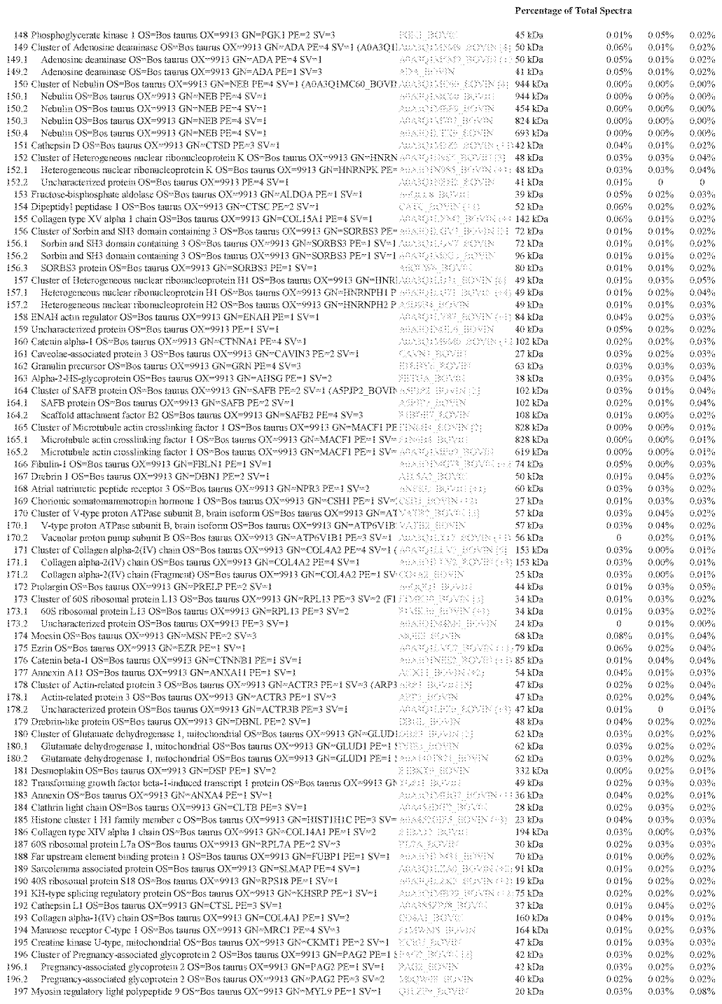

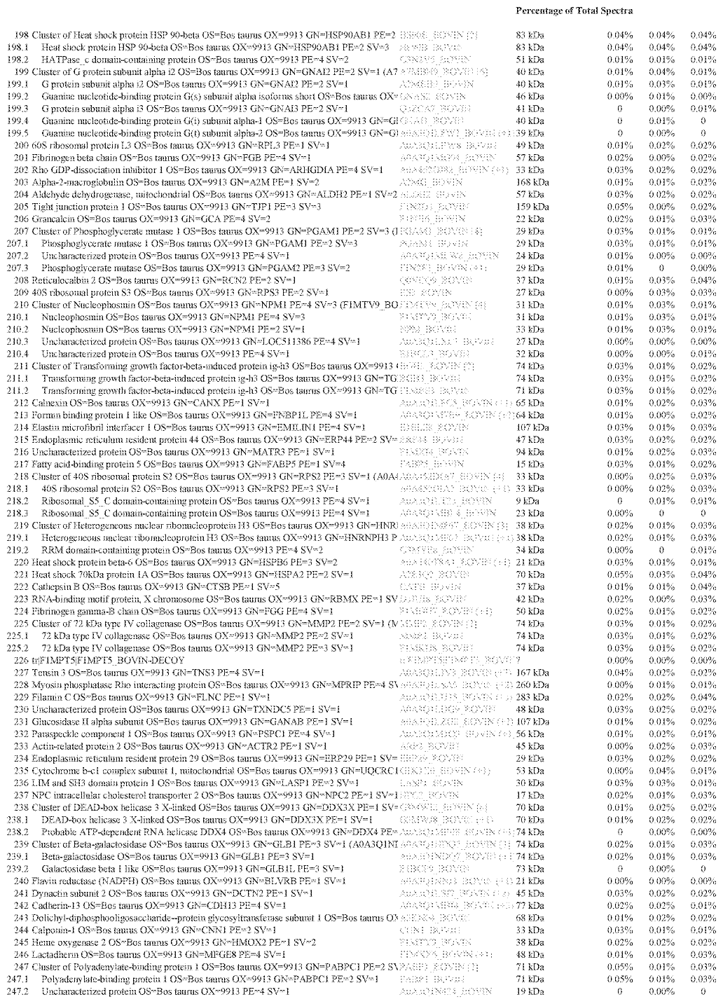

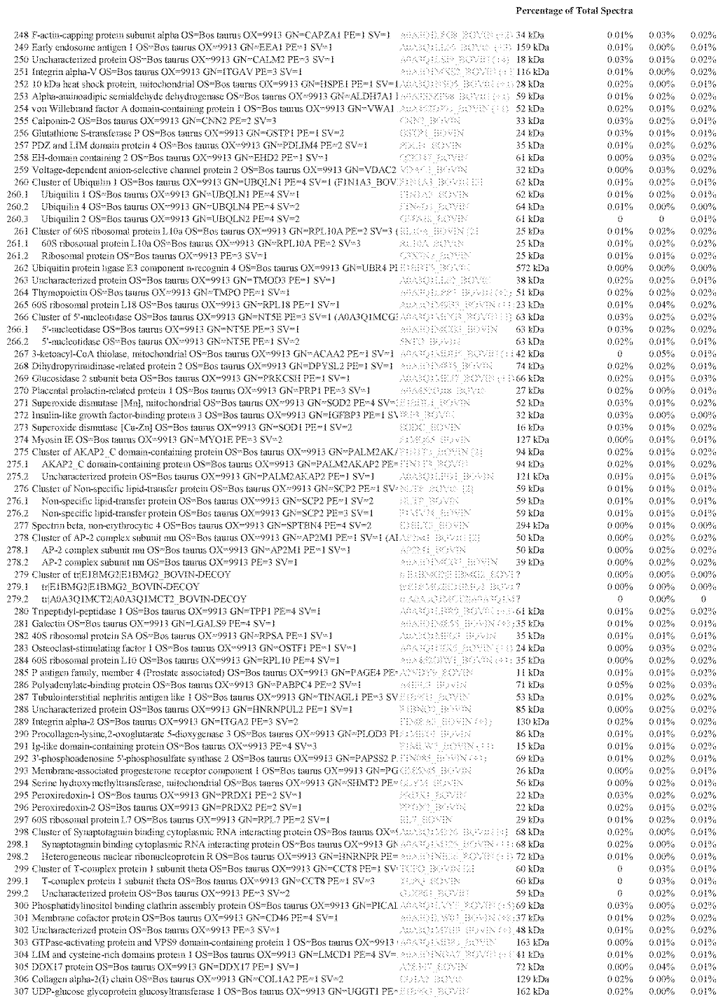

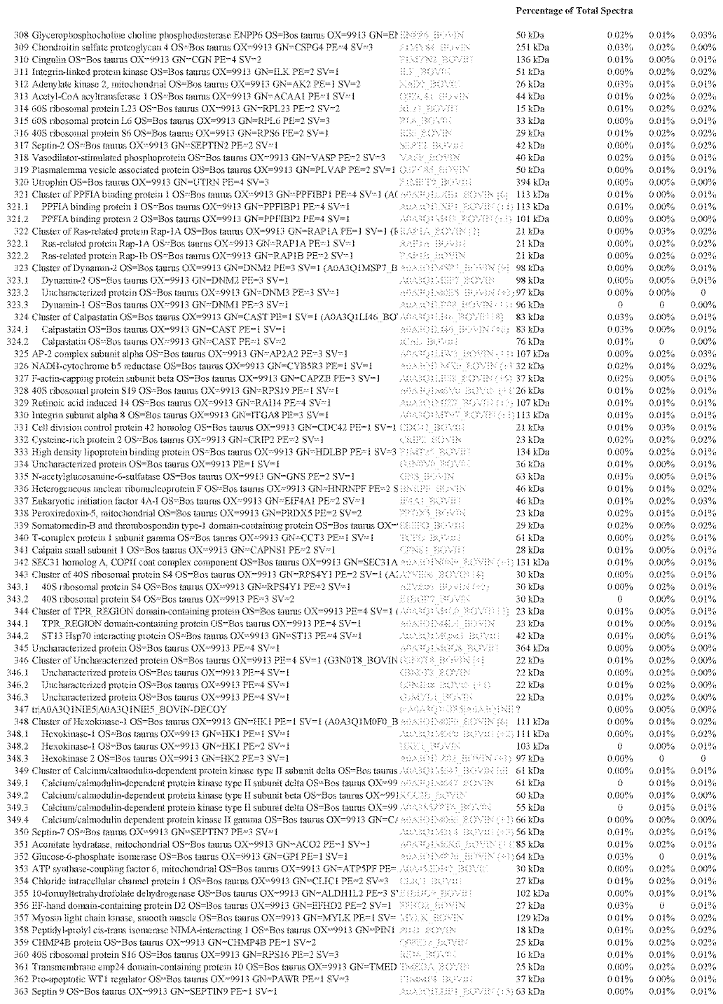

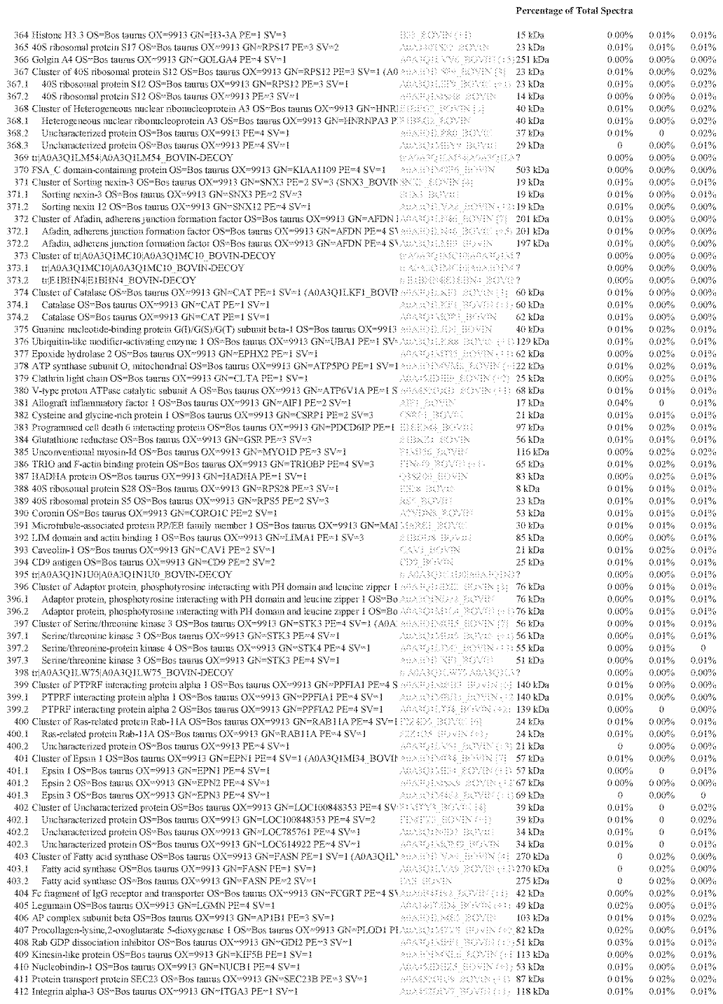

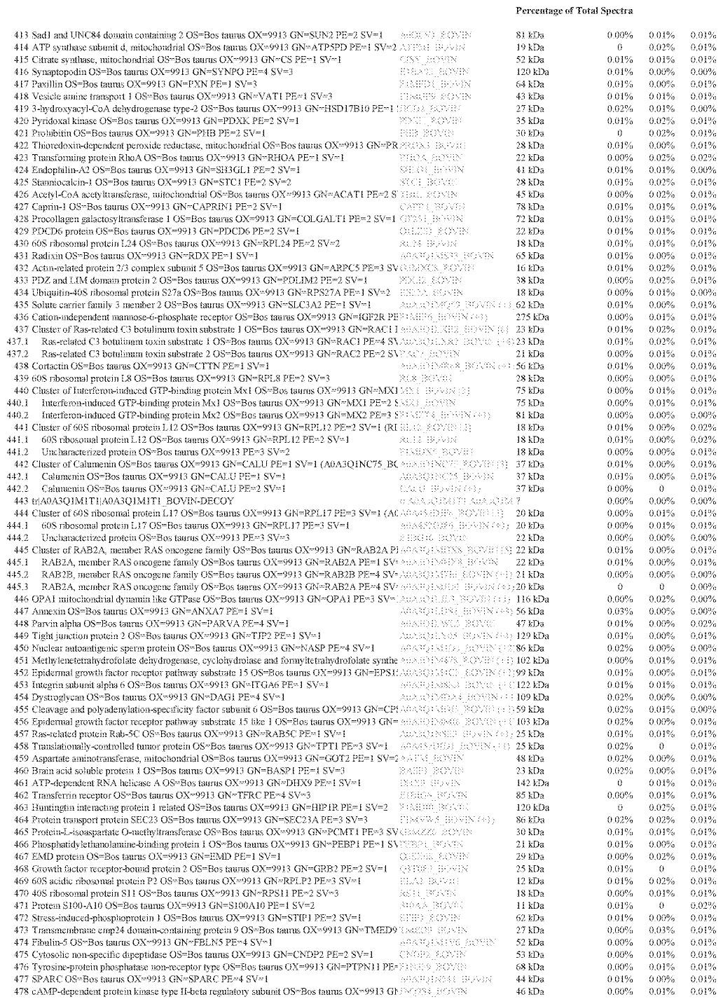

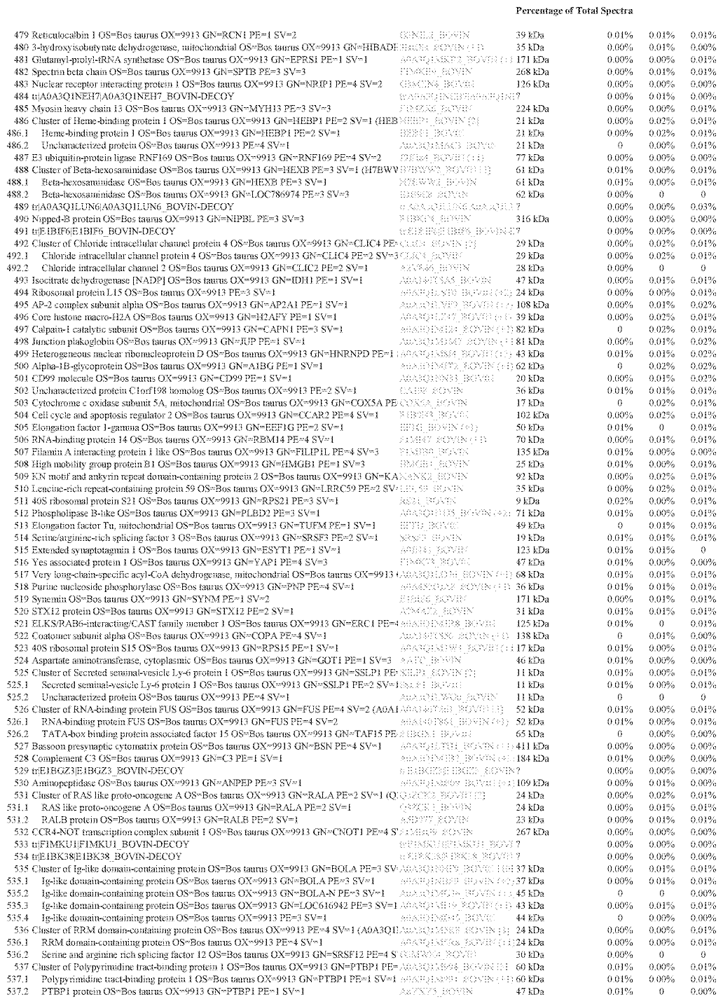

Referring to FIG. 7, bacterial load is mitigated both by decontamination of the placental tissue and through the process (i.e., process 100) of generating the placental extract composition (PE). FIG. 7 shows a stepwise drop in bacterial load at key stages in an embodiment of the placental extract composition process with a final reduction of 98.7%±2.4% (n=7) from ‘Extract process start’ (mechanically disrupting the tissues) to ‘Extract process final’ (after purifying the proteins). The example placental extract compositions used in the experiments depicted in FIG. 7 were obtained from process 100 wherein a bovine (cotyledonary) placenta was harvested in step 101.

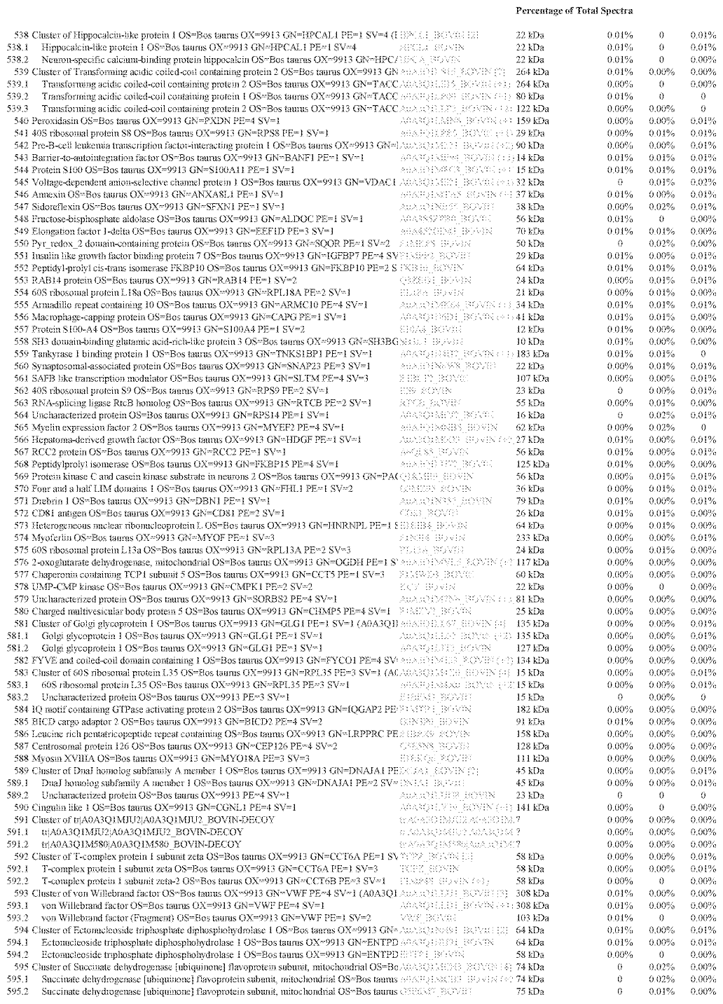

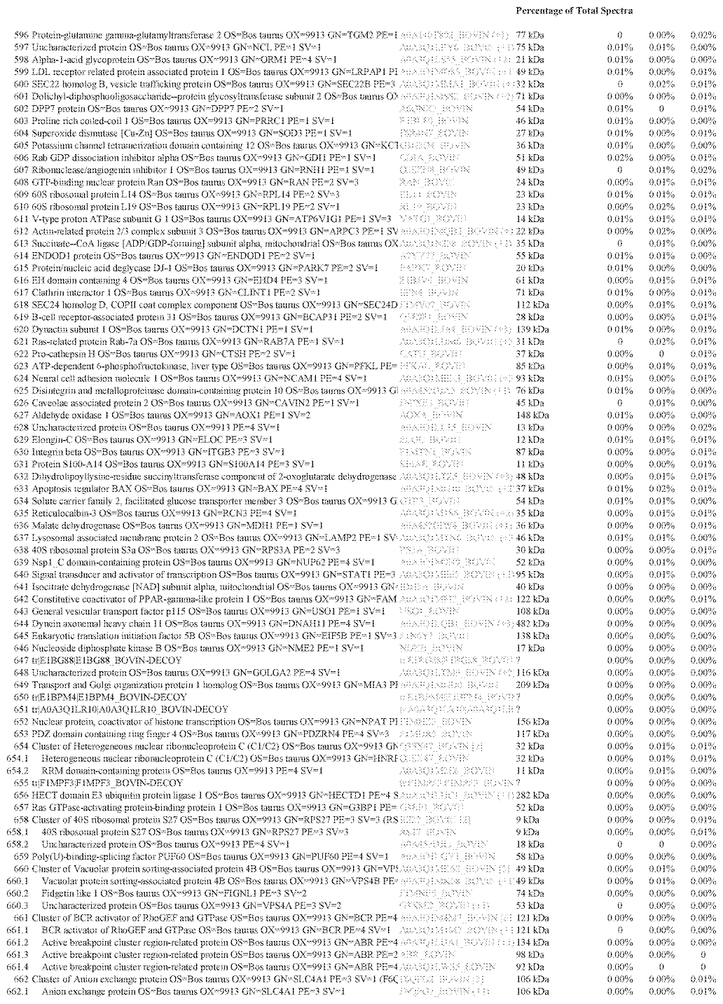

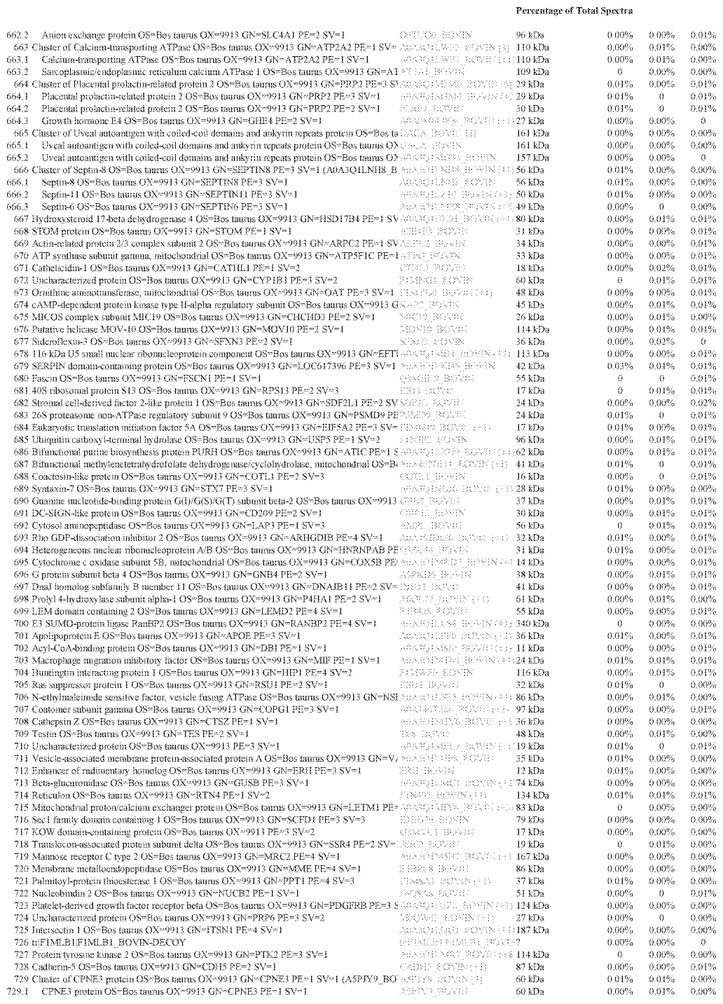

FIG. 8 shows the physical properties of a certain example embodiments of the invention. FIG. 8 demonstrates: (A) protein concentration; (B) pH of the placental extract composition; and (C) the change in urea content of the placental extract composition during the protein purification step. The particular placental extract composition (PE) is a high-protein liquid with a physiologically neutral pH and has an average protein concentration of 6.3 t 0.7 mg/mL, with no significant differences across 8 batches (NS=p=0.0833, n=3 per batch). The PE has an average pH of 7.33±0.03 (n=5). The particular PE used in these physical property experiments was the result of a method that includes a purification step (i.e., step 108 from process 100), during which the protein is retained and excess linearization reagents (urea) were removed (97.4%±2.6% of urea is removed C, n=8). Further, the placental extract compositions used in the experiments depicted in FIG. 8 were obtained from process 100 wherein a bovine (cotyledonary) placenta was harvested in step 101.

The biological properties of embodiments of placental extract compositions (PE) according to the invention have also been studied in vivo.

### Canine—Femoral Nonunion Study

The (canine) patient in this study was brought to a veterinary hospital after two unsuccessful attempts to surgically repair a broken femur at a different hospital. After these initial attempts, the patient presented with lameness and was non-weight bearing of the left hind limb. Repeat radiographs demonstrated a broken leg with a critical sized defect, lengthening bone plate, and a broken screw from the smaller plate. General physical examination demonstrated that the patient was in otherwise excellent condition.

Under general anesthesia, the surgical site was opened via a standard lateral approach to the left femur. Cultures of the surgical site were taken, and subsequent analysis was negative for aerobic growth. The fracture was then repaired via removal of the sequestrum (1 cm diameter), stabilization with a new bone plate (leg lengthening plate) and 3.5 mm bone plate screws, placement of a cancellous autograft with an embodiment of a placental extract composition (PE), and placement of gentamycin impregnated Kerrier beads around the surgical site. The cancellous autograft was taken bilaterally from the proximal shoulder and was mixed with 3 mL of the PE. This autograft-PE mixture was packed into the defect. The site was closed with 0 PDS simple continuous for deep fascia, 2-0 PDS simple continuous for superficial facia, and 3-0 PDS simple continuous for intradermal tissues. The remaining 2 mL PE was injected around the surgical site. Postoperative radiographs demonstrated appropriate reduction and fixation.

The placental extract composition used in this canine study was obtained from an embodiment of process 100 wherein a bovine (cotyledonary) placenta was harvested in step 101.

Despite two previous failures to heal, this procedure resulted in successful patient and radiographic outcomes. No evidence of complication was observed clinically or radiographically.

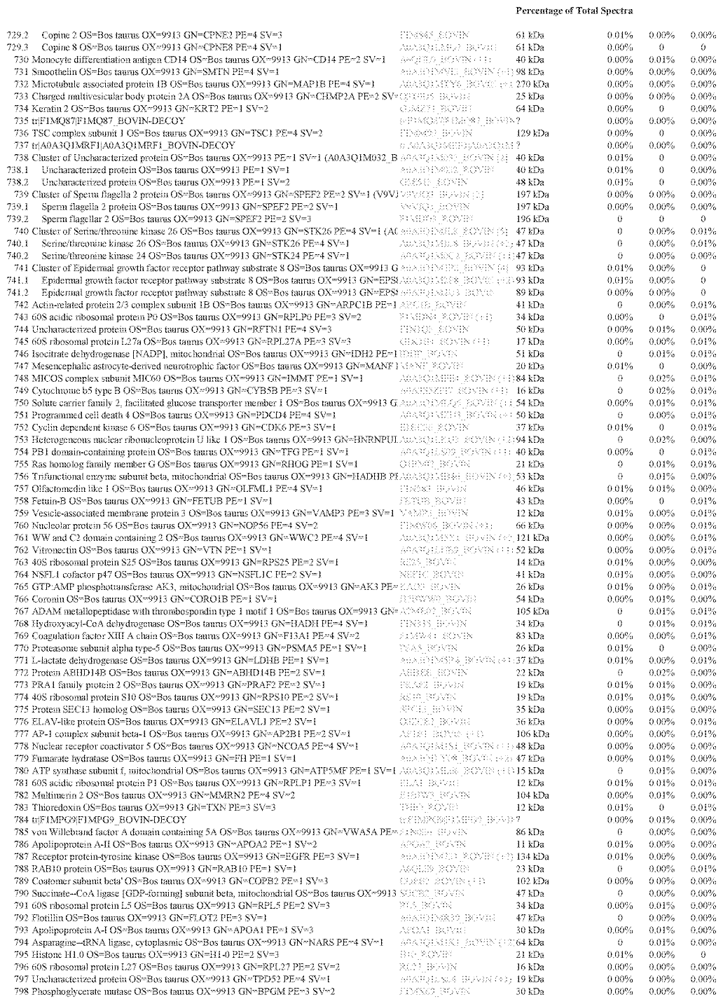

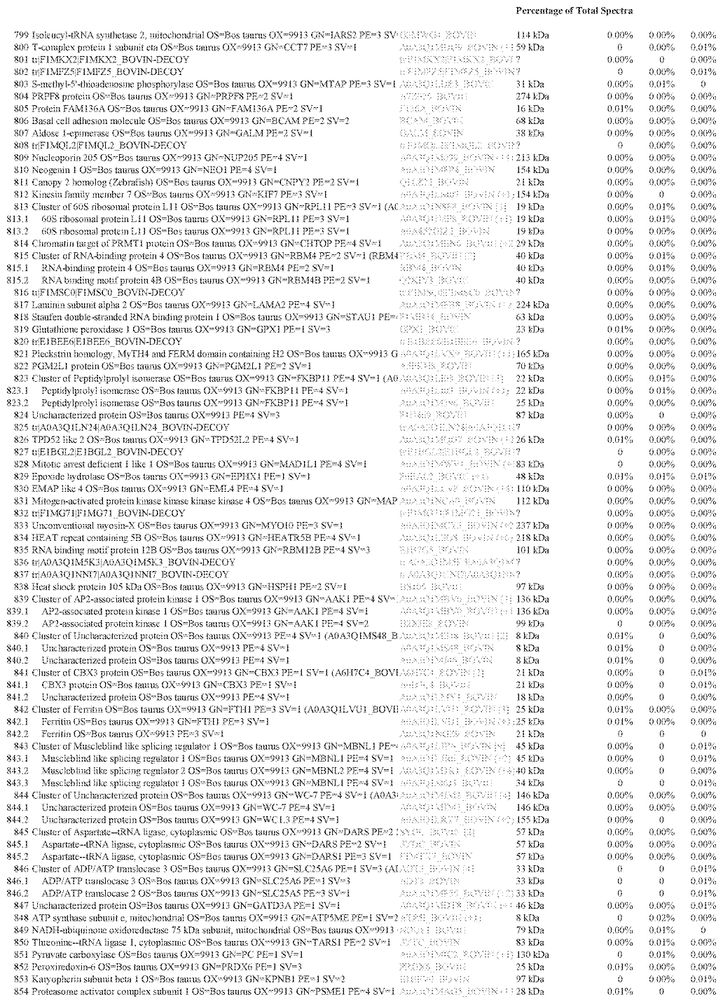

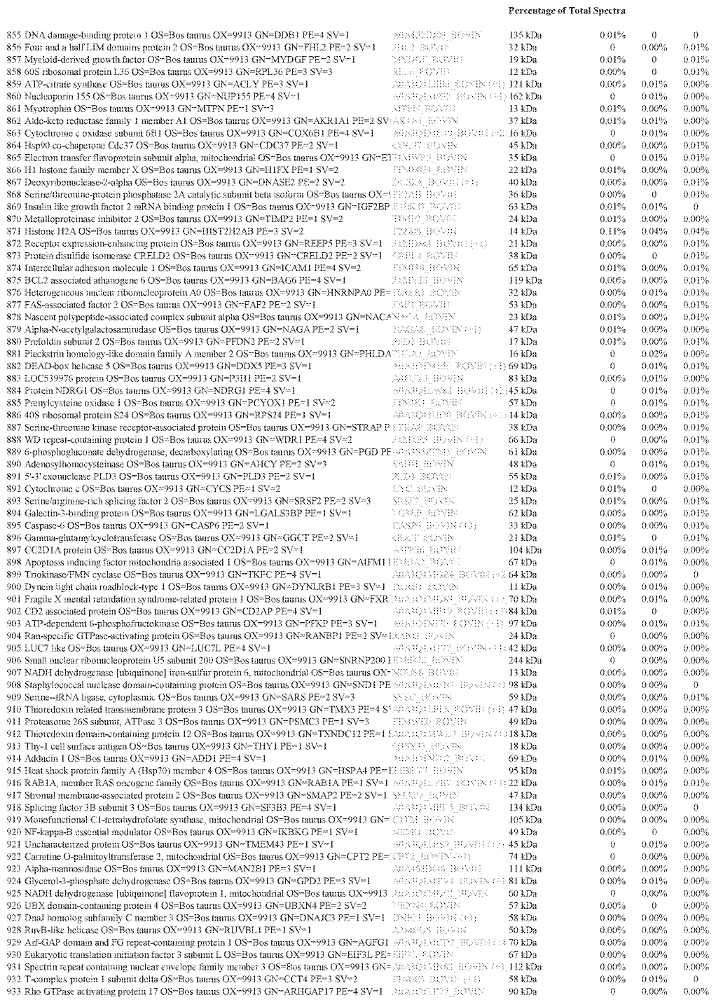

FIG. 9 illustrates the healing of the femoral non-union of the canine patient over a period of 24 weeks.

### Canine—Bone Arthrodesis

The (canine) patient was stepped on by a horse, leading to a broken leg. The patient was seen at a veterinary hospital and diagnosed with a fracture of the medial trochlear ridge of the talus of the left hind limb. The patient was treated with a cast for several months. Computed tomography confirmed a tarsal fracture.

Pan-tarsal arthrodesis was performed on the patient by osteotomy of the distal tibia and proximal talus and debridement of cartilage of the smaller joints. The tarsus was stabilized with a 3.5 broad locking bone plate. The joint spaces were packed with autogenous bone graft soaked with an embodiment of a placental extract composition (PE). Postoperatively the limb was placed in a bivalved cast.

The placental extract composition used in this canine study was obtained from an embodiment of process 100 wherein a bovine (cotyledonary) placenta was harvested in step 101.

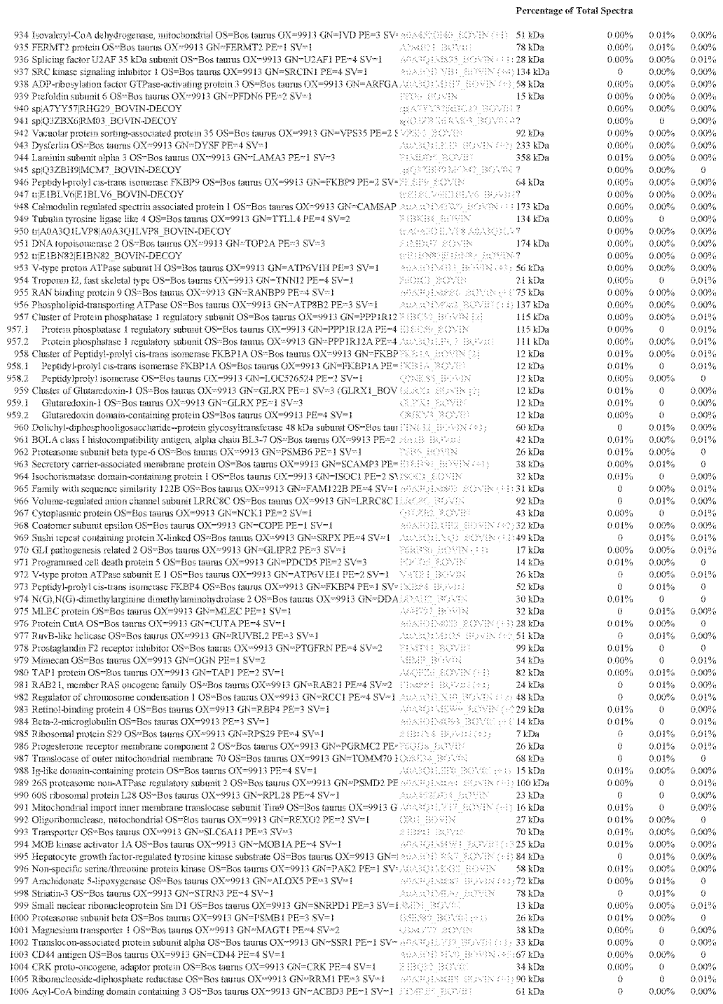

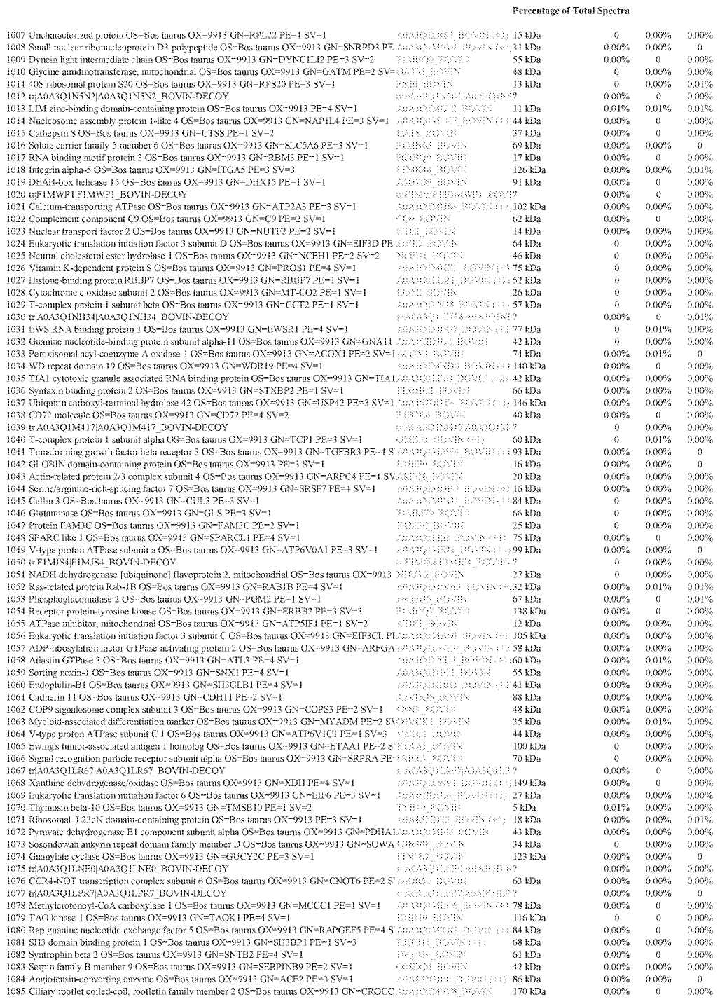

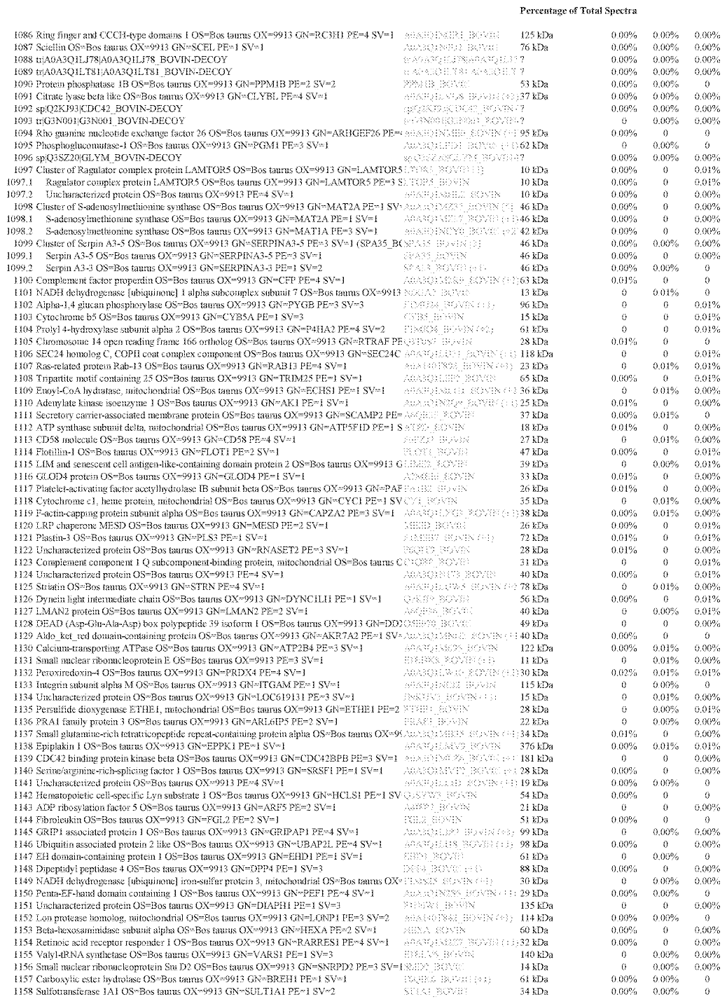

Following the procedure, no complications were observed clinically and as illustrated in FIG. 10, no complications were observed radiographically after seven weeks.

### Canine—Open Dermal Wound Study

An adult canine presented with a 4″×2″ wound on the left side of the head by the ear (Fig A). Observations of the wound included bleeding, redness, yellow-white discharge, and irritation. A veterinarian determined that the wound was a hot spot that had been scratched open. The wound remained open and unchanging for approximately I week, showing minimal signs of progress/healing. Within 3 days of the first observation, the wound size had increased.

Upon initial observation of the injury, the owner topically applied Neosporin and an anti-microbial spray twice daily. After 3 days, the wound worsened, and the patient was taken to a veterinary clinic. The veterinarian prescribed a pharmacological regimen including Cephalexin (antibiotic) 500 mg twice daily, Sivet L Spray 2-3 sprays every 8 hours, Apoquel (allergy medicine) 16 mg 1.5 tablets every 12 hours, and Proviable (probiotic) 1 capsule every 24 hours. Over the following 2.5 days of veterinarian-prescribed pharmacological treatment, there were minimal signs of improvement. At this time, a placental extract composition (PE) was then added to the wound site. For PE treatment, the hair around the area was trimmed and the irritated skin was washed with household soap and water. After patting the area dry, 2 ml, of PE was applied topically and rubbed into the wound with gloved hands. The PE was allowed to settle on the skin for approximately 5 minutes before the wound was bandaged in sterile gauze and medical tape. The gauze and medical tape was left on the patient for approximately 18 hours, after which the site was left open to the air. Following topical application of PE, the pharmacological regimen was continued.

The placental extract composition used in this canine study was obtained from an embodiment of process 100 wherein a bovine (cotyledonary) placenta was harvested in step 101.

Over the course of outpatient treatment during which only conventional pharmaceuticals were used, little-to-no improvement was noted on the patient. Despite the lack of progress in the preceding 5.5 days, within 18 hours of the topical PE application, the wound had fully scabbed over and was free of exudate or irritation. At this time, the owner reported a decreased perception of the patient's scratching frequency and discomfort. Less than a week following the PE application, the area was covered in a new bed of skin that was free of irritation or signs of infection. Within a month, visible recovery of hair over the area was observed.

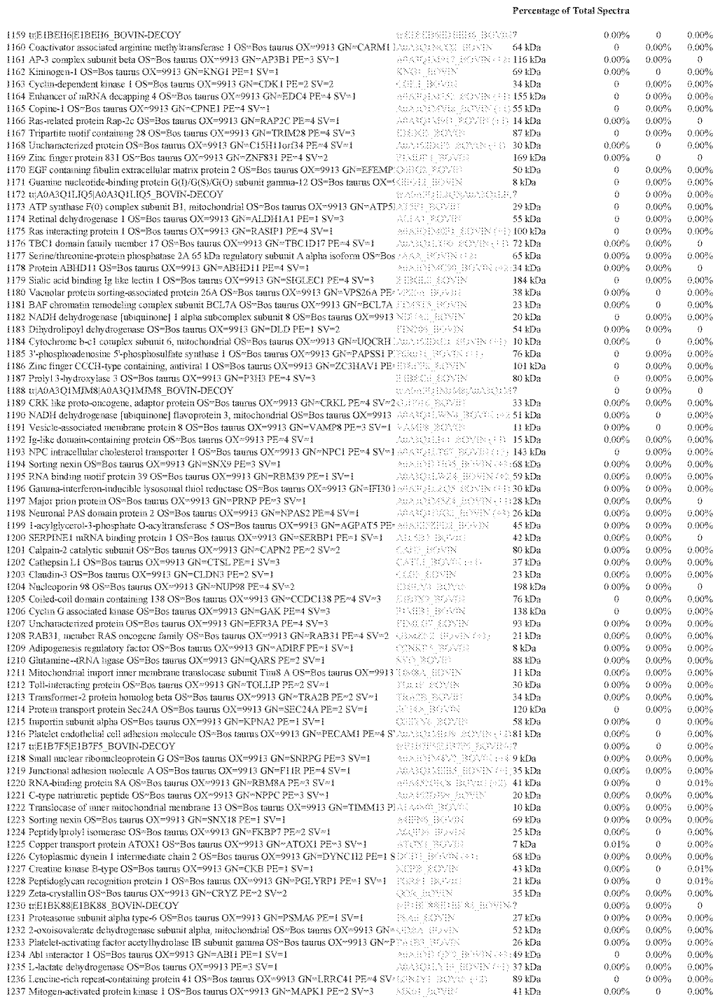

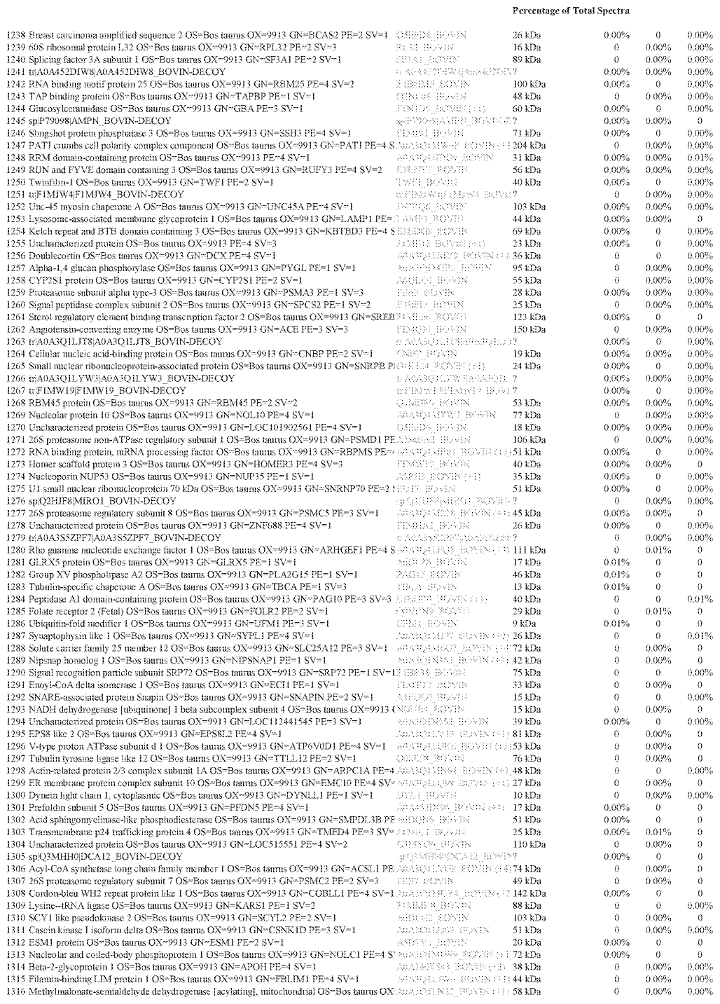

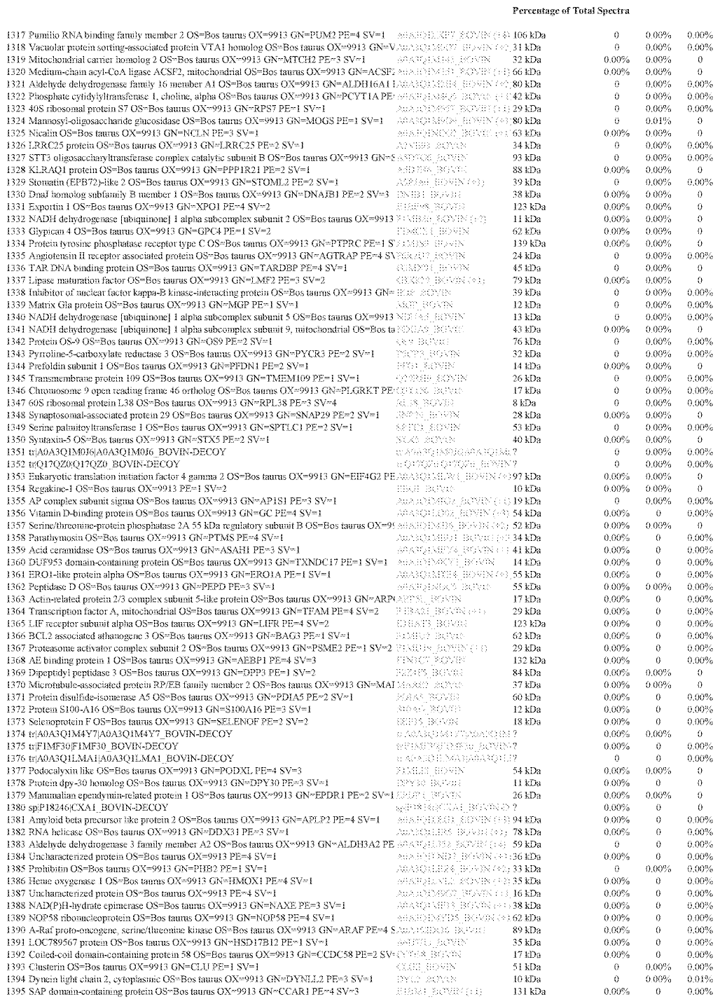

FIG. 11 illustrates the healing of the patient's skin wound showing: (A) the approximate location of the hot spot; (B) the initial appearance of the hotspot; (C) the appearance of the area after 5.5 days, the last 2.5 days of which included pharmacological treatment (the hair was trimmed at this time); (D) the appearance of the area 18 hours after topical application of PE, showing complete coverage of the skin; (E) the appearance of the area 5 days after treatment with PE, showing full closure of the skin; and (F) the appearance of the area 31 days after the initial treatment with PE, showing hair regrowth.

### Human—Burn Study #1

The (human) patient in this study was exposed to open flames for approximately 5 minutes. The resulting burn covered a majority of the patient's legs, lower abdomen, arms, hands and face. Physicians reported that the patient had fourth degree burns over 56% of the body. The patient was treated surgically, including debridement and grafting from the abdomen and back.

For approximately one year following discharge from the hospital, the patient followed a regimen in which Eucerin was applied topically. Over this time, the patient's burn sites closed, but scarring and discoloration persisted. The patient reported dissatisfaction and discomfort regarding the condition of the areas, particularly in the legs and ankles. At this time (1 year post discharge), the patient continued the traditional protocol but replaced the Eucerin with a formulation of placental extract composition (PE)—a cream. The PE cream was a formulation of PE in an emulsion of plant-based ingredients. The PE cream was applied to a defined area: the anterior face of the distal right leg. The cream was applied topically twice a day. This area was compared to the contralateral leg, which continued to be treated with Eucerin.

The placental extract composition used in this human study was obtained from an embodiment of process 100 wherein a bovine (cotyledonary) placenta was harvested in step 101.

Within one week of using the PE cream, the patient reported a reduction in the appearance of the scar coloration.

### Human—Burn Study #2

The (human) patient was exposed to an explosion that caused burns covering 18% of the body, primarily 3^{rd} degree burns localized to the distal lower extremities. Immediately after the explosion, the patient was treated with cold compresses and ice at a local hospital before being flown to a burn center for treatment. Physicians identified three critical sites: the right distal leg, exhibiting 3^{rd} degree burns, the left distal leg, exhibiting both 2^{nd} and 3^{rd} degree burns, and the right proximal leg, which would later serve as the autograft donor tissue.

Shortly after arriving at the burn center, the patient was given analgesics and necrotic tissue was removed. The patient underwent a total of 3 surgeries and remained at the burn center for 5 weeks for treatment and monitoring. The surgeries, occurring within 2 weeks of admittance, included (1) debriding and xenograft application to Site #2, (2) exchange of xenograft material, and (3) autografting from Site #3 to Site #1. While at the burn center, the patient had physical therapy daily and oxygen chamber therapy for 2 hours daily. The patient wore a custom, silicone-lined compression stocking for at least 23 hours per day on Site #1, continuing use after discharge from the hospital. After being discharged from the burn center, the patient followed the conventional burn regimen recommended by the hospital. This protocol involved daily washing with soap (Johnson and Johnson Baby Shampoo) and water, application of Eucerin Advanced Repair Cream, and avoidance of potential environmental stressors to the affected areas. The patient followed this regimen for 5 months.

After 5 months, when only moderate progress had been observed, the patient continued the traditional protocol but replaced the Eucerin with a formulation of placental extract composition (PE)—a cream. The PE cream was a formulation of PE in an emulsion of plant-based ingredients. The placental extract composition used in this human study was obtained from an embodiment of process 100 wherein a bovine (cotyledonary) placenta was harvested in step 101.

During the PE cream regimen, the patient did not use any other lotions or creams. The patient continued to use a compression stocking at Site #1 during this time.

The patient exhibited successful initial recovery: no infections were encountered, grafting material incorporated into the affected area, and a majority of the open wounds closed. However, in the 5 months following hospital discharge, the patient experienced pronounced scarring and scabbing. By month 6 (5 months after discharge), keloids had developed on the patient's ankle at Site #1. The patient expressed dissatisfaction and concern about the textural and visible condition of the sites. Despite the intensification of scarring over the first 5 months of using Eucerin, within 14 days of using the PE cream, the patient observed a visible reduction in scar coloration. By 2 months using of PE cream, skin softness, scar coloration, and keloid presence were pronouncedly improved.

### Human—Acne Lesion Study

The (human) patient was suffering from serious acne lesions and scarring for multiple years. The patient had tried Accutane for almost 2 years without any improvements to the skin.

The patient used a formulation of placental extract composition (PE)—a cream—on the face. The PE cream was applied topically once per evening after removing makeup and face washing. The PE cream was a formulation of PE in an emulsion of plant-based ingredients. The placental extract composition used in this human study was obtained from an embodiment of process 100 wherein a bovine (cotyledonary) placenta was harvested in step 101.

After thirteen days of use, the patient observed changes to the skin. After seven weeks of use, the patient's face showed a reduction of lesion quantity and intensity.

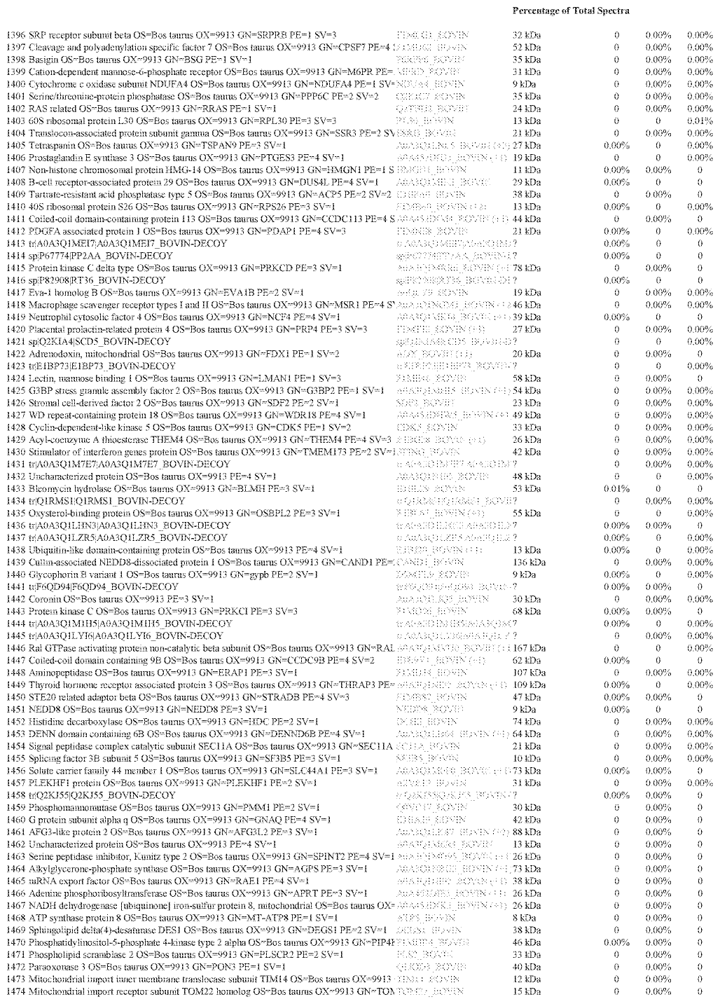

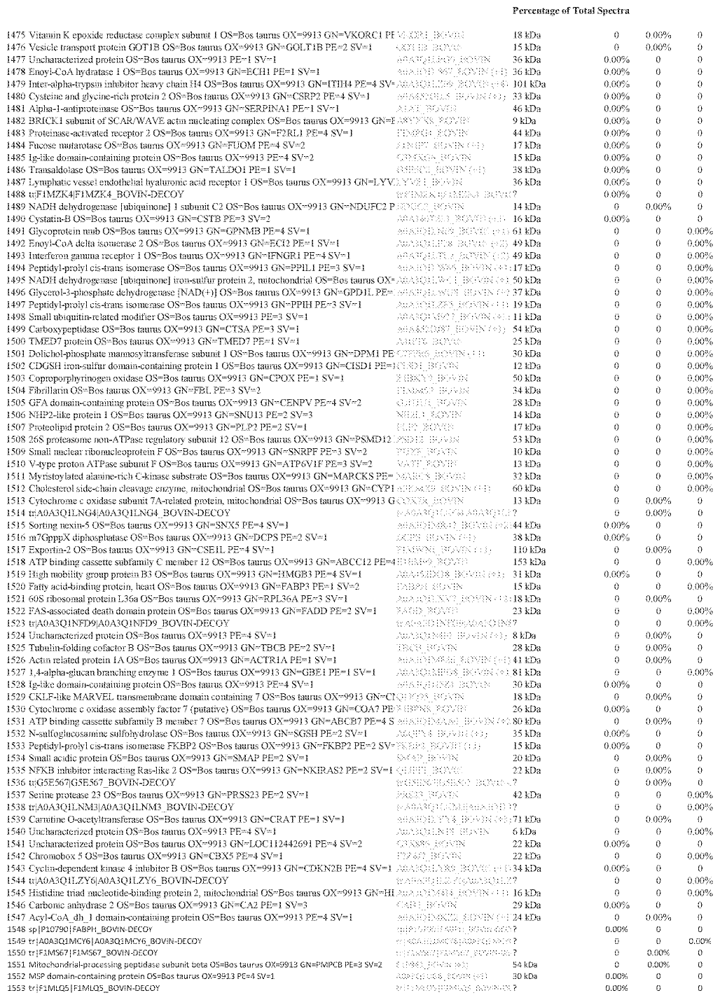

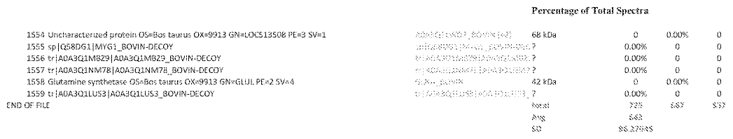

Embodiments of the placental extract composition have been analyzed by mass spectroscopy. For example, three lots of bovine placental extract composition prepared according to process 100 were analyzed by mass spectroscopy. The samples were run against an existing mass spectrometry library specific to the placenta species (i.e., bovine). The specific library used (Proteome ID UP 000009136, 9913 Bos Taurus, modified May 25, 2021) can be found at https://www.uniprot.org/proteomes/UP00009136. Three samples were analyzed and the range of proteins identified was 643±96.3. Of the 643 unique proteins (or protein fragments) identified, 594 had exact homolog matches in the human protein profile (92.4% match). The most prevalent proteins identified in this particular embodiment of the placental extract composition were clusters of vimentin and clusters of actin. FIG. 12 shows the results from the mass spectroscopy data for the three samples of the placental extract composition.

Examples of the proteins found in the sample placental extract composition included: actin, angiotensin-converting enzyme, angiotensinogen, annexins, collagens, desmin, elongins, epidermal growth factor, fibronectin, fibulin, heat shock proteins, helicases, heparan sulfate proteoglycan, hepatocyte growth factor, histones, insulin-like growth factor, keratin, laminins, myeloid-derived growth factor, myosin, neuroblast differentiation-associated protein, neurofilament medium polypeptide, plectin, potassium channel tetramerization domain, sodium/potassium-transporting ATPase, sorbins, superoxide dismutase, tenascin, transforming growth factor beta, troponins, tubulin, vitamin D binding protein, vitamin K epoxide reductase complex, zyxin (zinc-binding phosphoprotein).

Embodiments of cotyledonary (bovine) placental extract compositions prepared according to process 100 can have a total protein level ranging from about 1-10 mg/mL. Certain particular embodiments of cotyledonary (bovine) placental extract compositions have a total protein level of about 5 mg/mL.

Embodiment of placental extract compositions could be formulated for different treatments in a number of ways known in the art. In certain particular embodiments, the placental extract compositions can be applied topically or by injection as-is (as an unmodified liquid). In certain particular embodiments, placental extract compositions can be diluted in sterile buffers (e.g. for joint injections for osteoarthritis or I.M. injections for muscle atrophy). In certain particular embodiments, placental extract compositions can also be supplemented with various compounds relevant to the disease or tissue of interest (e.g. add a steroid or signaling molecule used therapeutically). In certain particular embodiments, placental extract compositions can be formulated by combining the composition with relevant carriers known in the art (e.g. added to bone grafts, added to hyaluronic acid solutions, added to ointments, added to collagen scaffolds). In certain particular embodiments, placental extract compositions can be formulated by combining with other treatments (e.g. added to cellular suspensions for injection, added to engineered tissues, added to autogenic cell cultures). In certain particular embodiments, placental extract compositions can also be formulated in different states (e.g. a powder that is added to a treatment site, a powder that is constituted in liquid before administration, a dried sheet that is moistened in situ or by adding sterile buffers). In certain particular embodiments, placental extract compositions can also be formulated to selectively remove specific components (e.g. filtered to remove large molecules such as collagen and hyaluronic acid).

The foregoing description is of certain preferred embodiments. Those of skill in the art will readily see variations and improvements that may be included in alternate embodiments in light of this disclosure. The invention described herein is not intended to be limited to the embodiments discussed in the detailed description or shown in the figures.

The additional disclosure below provides further description of embodiments of the present invention and related features according to the present disclosure. Also provided is further information regarding specific components that may be used in such embodiments. It will be understood that such further description and further information are for illustrative and exemplary purposes only and are not intended to be limiting.

1. A composition that is the result of the following steps:
obtaining cotyledonary placenta;
decontaminating the placenta;
mechanically disrupting the placenta;
placing the mechanically disrupted placenta in an ionic buffer with a protease neutralizing reagent to create a protein precipitate;
isolating and retaining the solid phase of the protein precipitate;
incubating the solid phase in a linearization buffer to linearize proteins;
isolating the soluble proteins by phase separation; and
purifying the liquid phase with soluble proteins by removing the linearization buffer to obtain a purified composition.

2. The composition of claim 1 wherein the cotyledonary placenta is one of the following:
bovine placenta, ovine placenta, or caprine placenta.

3. The composition of claim 1 wherein the cotyledonary placenta is bovine placenta.

4. The composition of claim 3 wherein the purified composition comprises between 647 and 740 unique proteins.

5. The composition of claim 3, wherein the two most prevalent protein clusters in the purified composition are vimentin and actin.

6. The composition of claim 1, wherein decontaminating the placenta comprises chemical treatment with water, bleach, iodine, isopropanol, sterile buffers, or a combination thereof, from which the washate is separated from the placenta via macropore filtration.

7. The composition of claim 1, wherein mechanically disrupting the placenta comprises grinding, chopping, cryo-milling, blending into pieces less than or equal to four square centimeters (≤4 cm²), probe homogenization, or a combination thereof.

8. The composition of claim 1, wherein the ionic buffer comprises sodium chloride, tris, phosphate buffered saline, saline, salt buffers with a molarity greater than or equal to three molar (≥3M), or a combination thereof.

9. The composition of claim 1, wherein placing the solid phase in a linearization buffer comprises incubating the solid phase with mechanical agitation at four degrees Celsius (4° C.).

10. The composition of claim 1, wherein the linearization buffer comprises urea, sodium dodecyl sulfate, chaotropic agents, detergents in buffers, or a combination thereof.

11. The composition of claim 1, wherein phase separation comprises centrifugation, filtration, or a combination thereof.

12. The composition of claim 1, wherein purifying the liquid phase with soluble proteins comprises dialyzing against a sterile buffer lacking linearizing reagents at four degrees Celsius (4° C.) for at least twelve cumulative hours (12 hr), dialyzing with a molecular weight cutoff of at least one kilo-Dalton (1 kDa), tangential flow filtration, or a combination thereof.

13. The composition of claim 1, wherein the steps further comprise sterilizing the composition to achieve a bioburden reduction of at least one log (1 log) via addition of a chemical sterilant, filtration, energy or particle irradiation, or a combination thereof.

14. The composition of claim 1, wherein the steps further comprise drying the composition by lyophilizing, powderizing the composition by cryo-milling, or a combination thereof.

15. The composition of claim 1, wherein the steps are conducted in a controlled environment of at least ISO level 8.

16. The composition of claim 13, wherein the steps are conducted in a controlled environment of at least ISO level 8.

17. The composition of claim 14, wherein the steps are conducted in a controlled environment of at least ISO level 8.

18. A method of making a composition comprising:
obtaining cotyledonary placenta;
decontaminating the placenta;
mechanically disrupting the placenta;
placing the mechanically disrupted placenta in a highly ionic buffer with a protease neutralizing reagent to create a protein precipitate;
isolating and retaining the solid phase of the protein precipitate;
placing the solid phase in a linearization buffer to linearize proteins;
isolating the soluble proteins by phase separation; and
purifying the liquid phase with soluble proteins by removing the linearization buffer to obtain a purified composition.

19. The method of claim 18 wherein the cotyledonary placenta is one of the following: bovine placenta, ovine placenta, or caprine placenta.

20. The method of claim 18 wherein the cotyledonary placenta is bovine placenta.

21. The method of claim 20 wherein the purified composition comprises between 647 and 740 unique proteins.

22. The method of claim 20, wherein the two most prevalent protein clusters in the purified composition are vimentin and actin.

23. The method of claim 18, wherein decontaminating the placenta comprises chemical treatment with water, bleach, iodine, isopropanol, sterile buffers, or a combination thereof, from which the washate is separated from the placenta via macropore filtration.

24. The method of claim 18, wherein mechanically disrupting the placenta comprises grinding, chopping, cryo-milling, blending into pieces less than or equal to four square centimeters (≤4 cm²), probe homogenization, or a combination thereof.

25. The method of claim 18, wherein the ionic buffer comprises sodium chloride, tris, phosphate buffered saline, saline, salt buffers with a molarity greater than or equal to three molar (≥3M), or a combination thereof.

26. The method of claim 18, wherein placing the solid phase in a linearization buffer comprises incubating the solid phase with mechanical agitation at four degrees Celsius (4° C.).

27. The method of claim 18, wherein the linearization buffer comprises urea, sodium dodecyl sulfate, chaotropic agents, detergents in buffers, or a combination thereof.

28. The method of claim 18, wherein phase separation comprises centrifugation, filtration, or a combination thereof.

29. The method of claim 18, wherein purifying the liquid phase with soluble proteins comprises, dialyzing against a sterile buffer lacking linearizing reagents at four degrees Celsius (4° C.) for at least twelve cumulative hours (12 hr), dialyzing with a molecular weight cutoff of at least one kilo-Dalton (1 kDa), tangential flow filtration, or a combination thereof.

30. The method of claim 18, wherein the steps further comprise sterilizing the composition to achieve a bioburden reduction of at least one log (1 log) via addition of a chemical sterilant, filtration, energy or particle irradiation, or a combination thereof.

31. The method of claim 18, wherein the steps further comprise drying the composition by lyophilizing, powderizing the composition by cryo-milling, or a combination thereof.

32. The method of claim 18, wherein the steps are conducted in a controlled environment of at least ISO level 8.

33. The method of claim 31, wherein the steps are conducted in a controlled environment of at least ISO level 8.

34. A method of treating a broken bone, burn, wound, or lesion in a mammal comprising: administering to the subject mammal a composition comprising a purified cotyledonary placental extract.

35. The method of claim 34 wherein the purified coytledonaly placental extract is one of the following: a purified bovine placental extract, a purified ovine placental extract, or a purified caprine placental extract.

36. The method of claim 34 wherein the purified coytledonaly placental extract is a purified bovine placental extract.

37. The method of claim 36 wherein the purified bovine placental extract comprises between 647 and 740 unique proteins.

38. The method of claim 36, wherein the two most prevalent protein clusters in the purified bovine placental extract are vimentin and actin.

39. The method of claim 34 wherein the subject mammal is a canine.

40. The method of claim 34 wherein the subject mammal is a human.

41. A method of inducing regenerative effects in a mammal comprising:
administering to the subject mammal a composition comprising a purified cotyledonary placental extract.

42. The method of claim 41 wherein the purified coytledonaly placental extract is one of the following: a purified bovine placental extract, a purified ovine placental extract, and a purified caprine placental extract.

43. The method of claim 41 wherein the purified coytledonaly placental extract is a purified bovine placental extract.

44. The method of claim 43 wherein the purified bovine placental extract comprises between 647 and 740 unique proteins.

45. The method of claim 43, wherein the two most prevalent protein clusters in the purified bovine placental extract are vimentin and actin.

46. The method of claim 43 wherein the subject mammal is a canine.

47. The method of claim 43 wherein the subject mammal is a human.

48. A method of inducing cell proliferative effects in a mammal comprising:
administering to the subject mammal a composition comprising a purified cotyledonary placental extract.

49. The method of claim 48 wherein the purified coytledonaly placental extract is one of the following: a purified bovine placental extract, a purified ovine placental extract, and a purified caprine placental extract.

50. The method of claim 48 wherein the purified coytledonaly placental extract is purified bovine placental extract.

51. The method of claim 50 wherein the purified bovine placental extract comprises between 647 and 740 unique proteins.

52. The method of claim 51, wherein the two most prevalent protein clusters in the purified bovine placental extract are vimentin and actin.

53. The method of claim 48 wherein the subject mammal is a canine.

54. The method of claim 48 wherein the subject mammal is a human.
